(19) United States
(12) Patent Application Publication
(10) Pub. No.: US 2017/0027530 A1
(43) Pub. Date: Feb. 2, 2017

(54) METHOD AND MEDICAL IMAGING APPARATUS FOR SPECIFYING AN EXAMINATION ENVIRONMENT FOR A MEDICAL IMAGING EXAMINATION

(71) Applicant: Siemens Healthcare GmbH, Erlangen (DE)

(72) Inventor: Bjoern Heismann, Erlangen (DE)

(73) Assignee: Siemens Healthcare GmbH, Erlangen (DE)

(21) Appl. No.: 15/223,165

(22) Filed: Jul. 29, 2016

(30) Foreign Application Priority Data
Jul. 30, 2015 (DE) 102015214482.8

(51) Int. Cl. A61B 5/00; A61B 5/055

(52) U.S. Cl. CPC A61B 5/7435; A61B 5/055; A61B 5/749

(57) ABSTRACT

In method for specifying an examination environment for a medical imaging examination, conducted by means of a medical imaging apparatus, at least one environment parameter of the examination environment of the medical imaging examination is selected by the patient via a selection unit that is accessible by the patient in the medical imaging apparatus. The at least one selected environment parameter is transferred to an environment adjustment processor, that specifies the examination environment on the basis of the at least one environment parameter.

### BACKGROUND OF THE INVENTION

Field of the Invention

The present invention concerns a method for specifying an examination environment for a medical imaging examination that is conducted by a medical imaging apparatus. The present invention further concerns a medical imaging apparatus having a patient receiving zone for receiving a patient for the purpose of a medical imaging examination.

Description of the Prior Art

A medical imaging examination conducted on a patient often triggers a stress situation in the patient. This can be attributable, for example, to cramped space conditions in the patient receiving zone of the medical imaging apparatus. A further cause can be very high levels of noise to which the patient is exposed during the medical imaging examination, particularly a magnetic resonance examination.

A stress situation of this type can lead to an increased sense of discomfort on the part of the patient. A feeling of discomfort or a negative emotional state of the patient can in turn lead to a deterioration in image quality, due for example to undesirable movements of the patient during the acquisition of medical image data. Furthermore, this can prolong a measurement duration of the medical imaging examination, in particular a magnetic resonance examination, because individual parts of the magnetic resonance examination have to be repeated due to movements of the patient.

### SUMMARY OF THE INVENTION

An object of the present invention is to provide a pleasant environment for the patient during a medical imaging examination.

The invention concerns a method for specifying an examination environment for a medical imaging examination conducted by a medical imaging apparatus that has the following steps.

At least one environment parameter of the examination environment of the medical imaging examination is selected by the patient via a selection unit that is accessible by the patient.

The at least one selected environment parameter is transferred from the selection unit to an environment adjustment processor.

The examination environment is specified by the environment adjustment processor on the basis of the at least one environment parameter.

As used herein, an examination environment means the environment of the patient during the medical imaging examination. During the medical imaging examination, the patient is preferably located inside a patient receiving zone of the medical imaging apparatus. Furthermore, an environment parameter means a parameter by which the examination environment can be influenced and/or specified. For example, the environment parameter can relate to how the patient receiving zone is illuminated. Moreover, the environment parameter can relate, for example, to a selection of music which the patient will listen to during the medical imaging examination.

The medical imaging apparatus can be any medical imaging considered appropriate by those skilled in the art. Preferably the medical imaging apparatus is a magnetic resonance apparatus because in this type of apparatus, due to the cramped space conditions inside the patient receiving zone and the relatively long duration of the examination, the present invention can be utilized with particular advantage.

The selection unit can be, for example a voice input unit or a mobile selection unit, such as a mobile telephone, in particular a smartphone, for example, or alternatively a touch display integrated inside the patient receiving zone. In order to specify the examination environment, the environment adjustment processor preferably has appropriate software and/or computer programs that, when executed in the environment adjustment processor, result in a specification of the examination environment. For this purpose the environment adjustment processor has at least one processor component and at least one memory.

Specifying the examination environment on the basis of the at least one environment parameter preferably is implemented by assigning a fixed value to the at least one environment parameter. This assignment of a fixed value to the at least one environment parameter causes a change in the examination environment, for example a change in a volume level and/or a change in a lighting level of the patient receiving zone and/or a change in a music selection, etc.

The method according to the invention enables a patient to change a setting of the examination environment prior to and/or during the examination and thereby adjust the examination environment directly or immediately to suit his or her wishes and/or his or her emotional state. This can help relax and/or calm the patient, as a result of which a high image quality can advantageously be achieved during the medical imaging examination and/or the measurement duration of the medical imaging examination can be shortened.

In an embodiment, the selection of the at least one environment parameter takes place during the medical imaging examination. In this way a patient can directly modify the environment during a medical imaging examination and thus adjust the examination environment immediately to suit his or her mood and/or his or her subjective perceptions. This can also prevent an unwanted interruption of the medical imaging examination, since the patient is able to adjust the examination environment first before any premature termination of the medical imaging examination becomes necessary.

Alternatively or in addition, the at least one environment parameter can be selected prior to the medical imaging examination. This has the advantage that undesirable movements of the patient for the purpose of selecting the at least one environment parameter can be avoided during the medical imaging examination. The selection can in this case be made just prior to the start of data acquisition in the medical imaging examination. For this purpose, the patient may already be situated inside the patient receiving zone of the medical imaging device and consequently can already adjust the examination environment according to his or her wishes.

In a further embodiment of the invention, the selection unit has a voice input unit and the at least one environment parameter is selected by a voice input by the patient. Thus it is possible to avoid an undesirable movement, in particular an undesirable hand movement, for the purpose of selecting the at least one environment parameter during a medical imaging examination. Furthermore, the patient in this situation can adjust the examination environment particularly quickly by selection of the at least one environment parameter. Preferably, the voice input unit is designed to register simple commands, in particular configuration commands, spoken by the patient, such as, for example, “More light!”, “Make it darker, please”, “More air!”, “Make airflow warmer!”, “Want to listen to music”, “Make couch warmer”, “Abort”, etc.

Alternatively or in addition, the selection unit can be a mobile selection unit, via which the at least one environment parameter can be selected. A mobile selection unit in this context is, for example, a mobile telephone, in particular a touchscreen of a smartphone, or a touchscreen of a tablet PC, etc. The different environment parameters can be visualized in this case by presentation of simple icons so that it is easily possible to select the at least one environment parameter. Moreover, it is also possible to make a voice-independent selection and thereby remove an additional stress factor from the patient during the medical imaging examination.

In a further embodiment of the invention, the at least one environment parameter can be a lighting parameter. This enables a lighting level, in particular illumination of the patient receiving zone, to be adjusted by the patient himself or herself to match his or her mood and/or his or her emotional state. In this context, a lighting parameter is to be understood as a parameter that can bring about a change in how the patient receiving zone is illuminated.

Alternatively or in addition, the at least one environment parameter can be a ventilation parameter. This enables ventilation, in particular ventilation of the patient receiving zone, to be adjusted by the patient himself or herself to match his or her mood and/or his or her emotional state. In this context a ventilation parameter is to be understood as a parameter that can bring about a change in how the patient receiving zone is ventilated.

Alternatively or in addition, the at least one environment parameter can be a temperature parameter. This enables a temperature, in particular the temperature of the patient receiving zone, to be adjusted by the patient himself or herself to match his or her mood and/or his or her emotional state, in particular to suit his or her current perception of the temperature. In this context a temperature parameter is to be understood as a parameter that can bring about a change in temperature of the patient receiving zone.

Alternatively or in addition, the at least one environment parameter can be an entertainment parameter. This enables, for example, a volume to be adjusted and/or a music selection and/or a film selection, etc., to be personalized by the patient himself or herself to suit his or her mood and/or his or her emotional state, in particular inside the patient receiving zone during the medical imaging examination. In this context an entertainment parameter is to be understood as a parameter that can bring about a change in an entertainment range of the patient, for example a volume setting and/or a music selection and/or a film selection, etc.

In a further embodiment of the invention, an abort parameter for prematurely terminating the medical imaging examination can be made via the selection unit. Particularly advantageously, a high level of safety or reassurance is provided for the patient because the patient knows that he or she can abort the medical imaging examination at any time if his or her condition, in particular a state of stress, no longer allows the medical imaging examination to be continued. In this context an abort parameter is to be understood in particular as a parameter which can cause the medical imaging examination to be aborted and/or terminated.

The invention furthermore relates to a medical imaging apparatus having a patient receiving zone for receiving a patient for the purpose of a medical imaging examination, wherein the medical imaging apparatus has a selection unit that is accessible by a patient, via which at least one environment parameter of an examination environment for a medical imaging examination is selected by the patient, an environment adjustment processor and a transmission unit that transfers the at least one environment parameter from the selection unit to the environment adjustment processor, and the environment adjustment processor is configured to specify the examination environment based on the at least one environment parameter.

The invention makes it possible for a patient to be able to change a setting of the examination environment prior to and/or during the examination and thereby adjust the examination environment directly or immediately to suit his or her wishes and/or his or her emotional state. This can help relax and/or calm the patient, as a result of which a high image quality can be achieved during the medical imaging examination, and/or the measurement duration of the medical imaging examination can be shortened.

The patient receiving zone is preferably at least partially enclosed by the scanner of the medical imaging apparatus. For example, the patient receiving zone can be embodied as a cylinder shape, with the patient receiving zone being cylindrically enclosed by the scanner.

The advantages of the medical imaging apparatus according to the invention substantially correspond to the advantages of the method according to the invention for specifying an examination environment for a medical imaging examination, as explained in detail above. Features, advantages and alternative embodiments mentioned with regard to the method are applicable to the apparatus as well.

In an embodiment of the apparatus, the selection unit is a voice input unit. As noted above, such a voice input unit is designed to register simple commands, in particular configuration commands, spoken by the patient, such as “More light!”, “Make it darker, please”, “More air!”, “Make airflow warmer!”, “Want to listen to music”, “Make couch warmer”, “Abort”, etc. Via the voice input unit, it is possible to avoid an undesirable movement, in particular an undesirable hand movement, for the purpose of selecting the at least one environment parameter during the medical imaging examination. Moreover, the patient in this case can adjust the examination environment particularly quickly by selection of the at least one environment parameter.

In a further embodiment of the inventive apparatus, the selection unit is a mobile selection unit. The different environment parameters can be visualized in this case by presentation of simple icons so that it is easily possible to select the at least one environment parameter. It is also possible to make a voice-independent selection and thereby remove an additional stress factor from the patient during the medical imaging examination.

In an embodiment, the selection unit is situated at least partially inside the patient receiving zone. This allows a particularly space-saving arrangement of the selection unit. Moreover, a good visibility of the selection unit can be ensured and consequently a movement of the patient's head, in particular during a medical imaging examination, can be avoided.

The invention also encompasses a non-transitory, computer-readable data storage medium encoded with programming instructions, that can be loaded directly into a memory of a programmable environment adjustment processor of a magnetic resonance apparatus, the programming instructions then causing the environment adjustment processor to execute the method according to the invention, as described above. The program code may require peripherals, e.g. libraries and help functions, in order to realize the corresponding embodiments of the method. The program code can form software having a source code that still needs to be compiled and linked or which only has to be interpreted, or an executable software code that only needs to be loaded into a computer in order to execute.

### BRIEF DESCRIPTION OF THE DRAWINGS

FIG. 1 schematically illustrates a medical imaging apparatus according to the invention.

FIG. 2 is a flowchart of the method according to the invention for specifying an examination environment for a medical imaging examination.

### DESCRIPTION OF THE PREFERRED EMBODIMENTS

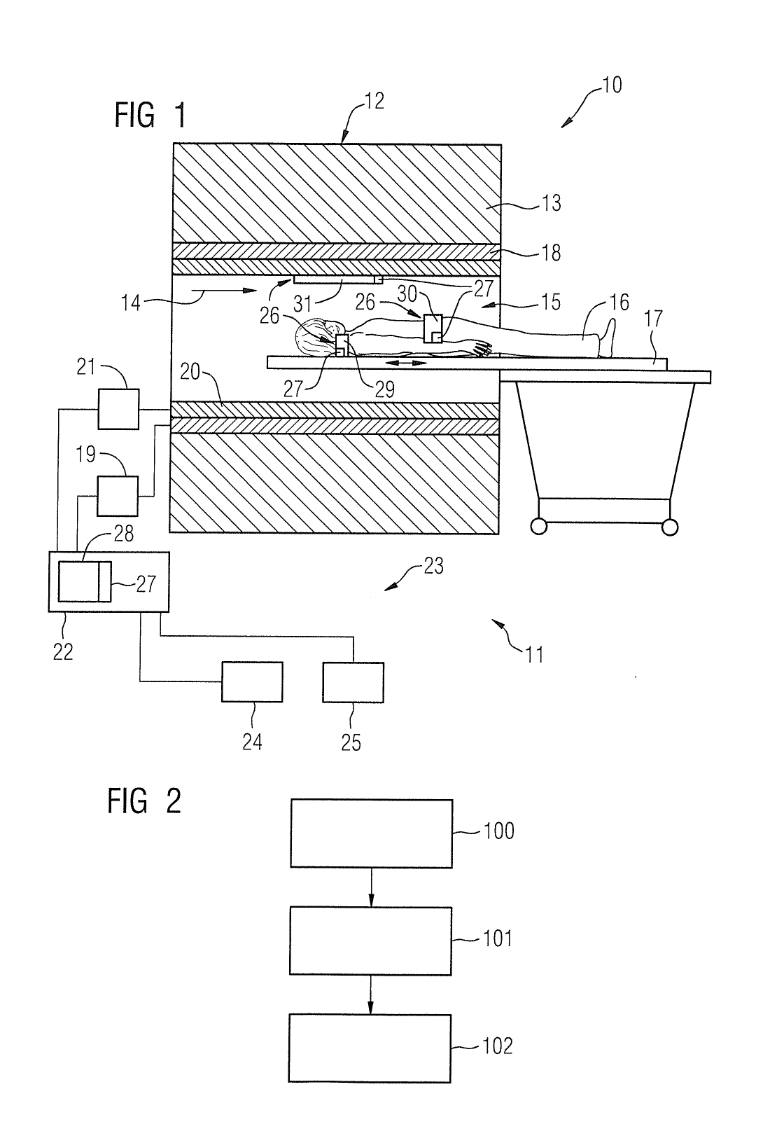

A medical imaging apparatus is shown schematically in FIG. 1. In the exemplary embodiment, the medical imaging apparatus is formed as a magnetic resonance apparatus 11, and the invention is explained using the example of the magnetic resonance apparatus 11. The present invention is not limited to the embodiment of the medical imaging apparatus as a magnetic resonance apparatus 11, and other embodiments of the medical imaging apparatus are conceivable.

The magnetic resonance apparatus 11 has a scanner 12 that has a superconducting basic field magnet 13 for generating a strong and constant basic magnetic field 14. The scanner 12 has a patient receiving zone 15 for receiving a patient 16. The patient receiving zone 15 in the exemplary embodiment is formed as a cylinder shape and is cylindrically enclosed by the scanner 12 in a circumferential direction. A different embodiment of the patient receiving zone 15 is conceivable. The patient 16 can be introduced into the patient receiving zone 15 by a patient support device 17 of the magnetic resonance apparatus 11.

The scanner 12 also has a gradient coil arrangement 18 for generating magnetic field gradients that are used for spatial encoding during an imaging session. The gradient coil arrangement 18 is controlled by a gradient control processor 19 of the magnetic resonance apparatus 11. The scanner 12 additionally has a radiofrequency antenna 20 for exciting nuclear spins in the patient 16 so as to deviate from the polarization that is established by the basic magnetic field 14. The radiofrequency antenna 20 is controlled by a radiofrequency antenna control processor 21 and radiates radiofrequency magnetic resonance sequences into an examination space that is substantially formed by the patient receiving zone 15.

The magnetic resonance apparatus 11 has a system control computer 22 that controls the basic field magnetic 13, the gradient control processor 19 and the radiofrequency antenna control processor 21. The system control computer 22 is responsible for the centralized control of the magnetic resonance apparatus 11, such as performing a predetermined imaging gradient echo sequence, for example. In addition, the system control computer 22 is configured to evaluate medical image data acquired during the magnetic resonance examination. The magnetic resonance apparatus 11 furthermore has a user interface 23 connected to the system control computer 22. Control information such as imaging parameters, as well as reconstructed magnetic resonance images can be displayed for the medical operating staff on a display monitor 24, for example on at least one screen, of the user interface 23. The user interface 23 also has an input unit 25 via which information and/or parameters can be entered by the medical operating staff during a measurement process.

In order to perform the method according to the invention for specifying an examination environment for a medical imaging examination, the magnetic resonance apparatus 11 additionally has a selection unit 26, a transmission unit 27 and an environment adjustment processor 28. Via the selection unit 26, the patient 16 selects at least one environment parameter of an examination environment for the magnetic resonance examination. To that end the selection unit 26 can have a voice input unit 29. Via the voice input unit 29, the at least one environment parameter is selected by a voice input. In addition, the selection unit 26 can have a mobile selection unit 30 formed as a smartphone and/or a tablet PC, the selection being made via a touch display of the smartphone and/or of a tablet PC.

The selection unit 26 is situated at least partially inside the patient receiving zone 15 as to be accessible by the patient 16. For this purpose, the selection unit 26 can have a display inside the patient receiving zone 15, in particular a touch display 31, on which the patient 16 can actuate his or her selection of the environment parameters.

In embodiments of the selection unit 26, the selection unit 26 has only a voice input unit 29 or only a mobile selection unit 30 or only a touch display 31, situated inside the patient receiving zone 15.

The transmission unit 27 is configured to transfer the at least one selected environment parameter from the selection unit 26 to the environment adjustment processor 28. In this case the transmission unit 27 can be at least partially integrated inside the selection unit 26 and/or inside the environment adjustment processor 28. The environment adjustment processor 28 is configured to specify the at least one environment parameter of the examination environment in response to the patient's selection.

The method according to the invention for specifying an examination environment for a medical imaging examination, in particular a magnetic resonance examination, is illustrated in FIG. 2, the magnetic resonance examination being conducted by means of the magnetic resonance apparatus 11. The method is performed by the environment adjustment processor 28 in combination with the selection unit 26. For this purpose, the environment adjustment processor 28 and/or the selection unit 26 have/has requisite software and/or computer programs configured to perform the method according to the invention when the software and/or computer programs are executed. The software and/or computer programs are stored in a memory of the environment adjustment processor 28 and/or of the selection unit 26, and are executed by a processor component of the environment adjustment processor 28 and/or of the selection unit 26 in order to perform the method for specifying an examination environment for a medical imaging examination.

In a first method step 100, a selection of at least one environment parameter or of two or more environment parameters of the examination environment of the medical imaging examination takes place. The at least one environment parameter is or the two or more environment parameters of the examination environment are selected by the patient 16 by means of the selection unit 26 so that the patient 16 can adjust the examination environment according to his or her wishes.

In this case the selection can be made with respect to time already prior to the medical imaging examination, in particular the magnetic resonance examination, so that a pleasant examination environment for the patient 16, in which the patient 16 feels at ease and is exposed to few stress factors, is already present at the start of the magnetic resonance examination. In addition, the selection can also be made during the magnetic resonance examination so that the patient 16 can adjust the examination environment to suit a current situation during the magnetic resonance examination.

In this case the selection can be made both by means of the voice input unit 29 of the selection unit 26 and by means of the mobile selection unit 30 or alternatively the touch display 28 arranged inside the patient receiving zone 15. The voice input unit 29 is in this case designed for registering voice commands and for generating at least one environment parameter on the basis of the registered voice commands. The voice commands preferably comprise simple commands, in particular configuration commands spoken by the patient 16, such as, for example, “More light!”, “Make it darker, please”, “More air!”, “Make airflow warmer!”, “Want to listen to music”, “Make couch warmer”, “Abort”, etc.

Preferably, the selection of the one environment parameter or of the two or more environment parameters of the examination environment is made by means of the voice input unit 29 during the magnetic resonance examination, since in this situation a movement of the patient 16 is undesirable. Furthermore, the patient 16 can also select the one environment parameter and/or the two or more environment parameters for setting and/or specifying the examination environment during the magnetic resonance examination by means of the selection unit arranged inside the patient receiving zone 15, in particular by means of the touch display 31 arranged inside the patient receiving zone 15. In addition, the mobile selection unit 30 is also available to the patient 16 for the selection of the one environment parameter or of the two or more environment parameters of the examination environment.

Preferably, the selection of the one environment parameter or of the two or more environment parameters of the examination environment is made by means of the mobile selection unit 30 prior to the magnetic resonance examination, since in this situation the patient 16 can make a selection of a number of environment parameters particularly quickly. Alternatively or in addition, the patient 16 can actuate the selection of the one environment parameter or of the two or more environment parameters of the examination environment prior to the magnetic resonance examination also by the voice input unit 29 and/or by the touch display 31 arranged inside the patient receiving zone 15.

The different environment parameters can be visualized on the touch display 31 arranged inside the patient receiving zone 15 and/or on the touch display of the mobile selection unit 30 by means of a presentation of simple icons so that it is easily possible for the patient 16 to select the one environment parameter or the two or more environment parameters on the selection unit 26. Moreover, in this situation the different environment parameters can also be selected for example by moving a displayed setting element along a setting range for at least one of the environment parameters.

In an alternative embodiment of the selection unit 26, the selection can be made exclusively by means of the voice input unit 29 or exclusively by means of a mobile selection unit 30 or exclusively by means of the touch display 31 arranged inside the patient receiving zone 15.

In this case the one selected environment parameter or the two or more environment parameters can be a lighting parameter. This enables the patient 16 to specify a lighting level and/or an illumination of the patient receiving zone 15. By selecting the lighting parameter the patient 16 can adjust the lighting and/or an illumination of the patient receiving zone 15 to suit his or her mood and/or his or her emotional state. In this case the lighting parameter is a parameter that can bring about a change in how the patient receiving zone 15 is illuminated. For example, a lighting level of the patient receiving zone 15 can be varied and/or set and/or specified by the lighting parameter.

Alternatively or in addition, the one environment parameter or the two or more environment parameters can be a ventilation parameter. This enables the patient 16 to specify a ventilation of the patient receiving zone 15. By selecting the ventilation parameter the patient 16 can adjust the ventilation of the patient receiving zone 15 to suit his or her mood and/or his or her emotional state. For example, a ventilation level of the patient receiving zone 15 can be varied and/or set and/or specified by the ventilation parameter.

Alternatively or in addition, the one environment parameter or the two or more environment parameters can be a temperature parameter. This enables the patient 16 to specify a temperature of the patient receiving zone 15. By selecting a temperature parameter the patient 16 can adjust the temperature of the patient receiving zone 15 to suit his or her mood and/or his or her emotional state. For example, a heat level of the patient receiving zone 15 can be varied and/or set and/or specified by the temperature parameter by, for example a temperature of the patient support device 17 being selected.

Alternatively or in addition, the one environment parameter or the two or more environment parameters can be an entertainment parameter. This enables a volume to be adjusted and/or a music selection and/or a film selection, etc., to be personalized by the patient 16 him- or herself in particular inside the patient receiving zone 15 during the medical imaging examination to suit his or her mood and/or his or her emotional state.

In a further method step 101, the one selected environment parameter and/or the two or more selected environment parameters are transferred from the selection unit 26 to the environment adjustment processor 28 by the transmission unit 27. In a further method step 102, the examination environment is specified in the environment adjustment processor 28 by means of the one selected environment parameter or the two or more selected environment parameters. Specifying the examination environment on the basis of the at least one environment parameter preferably includes assigning a fixed value to the at least one environment parameter, wherein the assignment of a fixed value to the at least one environment parameter preferably brings about a change in the examination environment.

Furthermore in the method according to the invention, in the first method step 100 of selecting the at least one environment parameter, an abort parameter for prematurely terminating the medical imaging examination can also be selected by the patient 16 via the selection unit 26. This enables the patient 16 to abort the magnetic resonance examination at any time if a condition of the patient 16 no longer allows a continuation of the magnetic resonance examination.

By the specification of the examination environment by the patient 16, the patient 16 can adjust the examination environment according to his or her wishes and/or his or her condition, in particular his or her emotional state, and in this way increase a sense of well-being during the medical imaging examination, in particular the magnetic resonance examination.

Although modifications and changes may be suggested by those skilled in the art, it is the intention of the inventor to embody within the patent warranted hereon all changes and modifications as reasonably and properly come within the scope of his contribution to the art.

I claim as my invention:

1. A method for specifying an examination environment for a medical imaging examination conducted by a medical imaging apparatus, said method comprising:
selecting at least one environment parameter of the examination environment of the medical imaging examination with a selection made by a patient within the medical imaging apparatus via a selection unit that is accessible to the patient from within the medical imaging apparatus;
transferring the at least one selected environment parameter from the selection unit to an environment adjustment processor; and
with said environment adjustment processor, specifying the examination environment dependent on said at least one environment parameter.

2. A method as claimed in claim 1 comprising selecting said at least one environment parameter during said medical imaging examination.

3. A method as claimed in claim 1 comprising selecting said at least one environment parameter prior to said medical imaging examination.

4. A method as claimed in claim 1 wherein said selection unit is a voice input unit, and comprising selecting said at least one environment parameter by a voice input made by the patient into said voice input unit.

5. A method as claimed in claim 1 wherein said selection unit comprising using a mobile selection unit as said selection unit.

6. A method as claimed in claim 1 wherein said at least one environment parameter is a lighting parameter.

7. A method as claimed in claim 1 wherein said at least one environment parameter is a ventilation parameter.

8. A method as claimed in claim 1 wherein said at least one environment parameter is a temperature parameter.

9. A method as claimed in claim 1 wherein said at least one environment parameter is an entertainment parameter.

10. A method as claimed in claim 1 comprising making an abort command, as said at least one environment parameter and, from said environment adjustment processor, terminating said medical examination in response to said abort command.

11. A medical imaging apparatus comprising:
a medical image data acquisition scanner having a patient receiving zone therein adapted to receive a patient for implementing a medical imaging examination;
a selection unit, accessible to the patient from within the medical imaging apparatus, configured to receive or detect at least one environment parameter of the examination environment of the medical imaging examination, as a selection made by a patient within the medical imaging apparatus;
an environment adjustment processor;
a transfer unit configured to transfer the at least one selected environment parameter from the selection unit to said environment adjustment processor; and
said environment adjustment processor being configured to specify the examination environment dependent on said at least one environment parameter.

12. A medical imaging apparatus as claimed in claim 11 wherein said selection unit is a voice input unit.

13. A medical imaging apparatus as claimed in claim 11 wherein said selection unit is a mobile selection unit.

14. A medical imaging apparatus as claimed in claim 11 wherein said selection unit is situated at least partially inside said patient receiving zone.

15. A non-transitory, computer-readable data storage medium encoded with programming instructions, said storage medium being loaded into an environment adjustment processor of a medical imaging apparatus, said medical imaging apparatus comprising a medical image data acquisition scanner adapted to receive a patient for implementing a medical imaging examination, and said medical imaging apparatus comprising a selection unit that is accessible by the patient in the scanner, said programming instructions causing said environment adjustment processor to:
receive at least one environment parameter of the examination environment of the medical imaging examination as a selection made by a patient within the medical imaging apparatus via the selection unit; and
specify the examination environment dependent on said at least one environment parameter.
